(19) United States
(12) Patent Application Publication
(10) Pub. No.: US 2013/0211485 A1
(43) Pub. Date: Aug. 15, 2013

(54) Probe Device and a Method of Fabricating the Same

(71) Applicant: AGENCY FOR SCIENCE, TECHNOLOGY AND RESEARCH, (US)

(72) Inventors: Anupama Vijay GOVINDARAJAN, Singapore (SG); Guang Kai Ignatius TAY, Singapore (SG); Woo-Tae PARK, Singapore (SG)

(73) Assignee: AGENCY FOR SCIENCE, TECHNOLOGY AND RESEARCH, Singapore (SG)

(21) Appl. No.: 13/766,042

(22) Filed: Feb. 13, 2013

(30) Foreign Application Priority Data
Feb. 13, 2012 (SG) SG201200998-1

(51) Int. Cl. A61N 1/05

(52) U.S. Cl. CPC A61N 1/05
USPC 607/116; 156/60; 156/272.2

(57) ABSTRACT

According to embodiments of the present invention, a probe device is provided. The probe device includes a flexible probe that is implantable into a biological tissue, a rigid carrier, and a biodegradable material received in a recess defined between the flexible probe and the rigid carrier, the biodegradable material bonding the flexible probe and the rigid carrier to each other, wherein the biodegradable material is dissolvable in the biological tissue such that the flexible probe and the rigid carrier are separable and the rigid carrier is removable from the biological tissue, and wherein the biodegradable material is capable of drug delivery upon dissolution. According to further embodiments of the present invention, a method of fabricating a probe device is also provided.

### CROSS-REFERENCE TO RELATED APPLICATION

This application claims the benefit of priority of Singapore patent application No. 201200998-1, filed Feb. 13, 2012, the contents of which are incorporated herein by reference for all purposes.

### TECHNICAL FIELD

Various embodiments relate to a probe device and a method of fabricating the probe device.

### BACKGROUND

Microelectromechanical systems (MEMS) devices are particularly promising as neuronal recording tools due to their small dimensions, suitability for multi-electrode recording, integration with on-chip electronics, and implantability. Research has successfully demonstrated electrical microprobes made from silicon that are suitable for neuronal recording. These silicon microprobes with typical cross-sections of tens of micrometers can penetrate living tissue without causing significant trauma.

MEMS devices for extracellular neuronal recording are at a point where structures capable of detailed mapping (acute and semi chronic) in the central nervous system (CNS) can be fabricated. With the success of the work on cochlear and Parkinson's [deep brain stimulation (DBS)] implants, it is clear that extracellular MEMS neuronal intervention devices have great promise. However, the mechanical mismatch between the stiff probe and the soft biological tissue can aggravate inflammation at the implantation site.

Micromotion is the relative movement between the implant and the brain tissue. Brain pulsations can be attributed to changes in intracranial pressure due to breathing and the cardiac pulse. For implants which are tethered to the skull, micromotion can also result from relative movement between the brain and the skull. While silicon has a Young's modulus of around 170 GPa, brain tissue has a Young's modulus of ˜3 KPa. This large mismatch in stiffness can contribute to shear induced inflammation at the implantation site. This inflammation encourages the formation of a glial scar, which can encapsulate the probe in time, isolating the electrode from the surrounding neural tissue. Polymer materials like Polyimide (Young's modulus ˜3 GPa), Parylene (Young's modulus ˜4 GPa), Su-8 (Young's modulus ˜2 GPa) and PDMS (Young's modulus ˜3 GPa) have been employed as the structures of flexible probes because they can fit into tissues and deform their shapes as the organs deform. Moreover, flexible electrodes may avoid both a gradual shift in the recording location and a reduction in the signal to noise ratio; these problems often occur with rigid electrodes during long term experiments because of the continued differential motion in tissue. However, the implantations with flexible neural probes is more challenging compared to conventional silicon (Si) neural probes and often requires the aid of a special insertion device or a pre-made hole at the insertion site, as implantation using traditional techniques is difficult due to their increased flexibility.

There are also conventional biodegradable backbone/dissolving core based neural probes. However, these have unknown issues of long term effects of the biodegradable core material being left in the cortex, where the amount of material being left in the cortex will be large as the biodegradable core material is the bulk of the neural probe.

### SUMMARY

According to an embodiment, a probe device is provided. The probe device may include a flexible probe that is implantable into a biological tissue, a rigid carrier, and a biodegradable material received in a recess defined between the flexible probe and the rigid carrier, the biodegradable material bonding the flexible probe and the rigid carrier to each other, wherein the biodegradable material is dissolvable in the biological tissue such that the flexible probe and the rigid carrier are separable and the rigid carrier is removable from the biological tissue, and wherein the biodegradable material is capable of drug delivery upon dissolution.

According to an embodiment, a method of fabricating a probe device is provided. The method may include forming a flexible probe that is implantable into a biological tissue, forming a rigid carrier, and bonding the flexible probe and the rigid carrier to each other with a biodegradable material between the flexible probe and the rigid carrier, wherein the biodegradable material is received in a recess defined between the flexible probe and the rigid carrier, wherein the biodegradable material is dissolvable in the biological tissue such that the flexible probe and the rigid carrier are separable, and wherein the biodegradable material is capable of drug delivery upon dissolution.

According to an embodiment, a probe device is provided. The probe device may include a flexible probe that is implantable into a biological tissue, a rigid carrier, and polyethylene glycol or sugar candy between the flexible probe and the rigid carrier bonding the flexible probe and the rigid carrier to each other, in which the polyethylene glycol or the sugar candy is dissolvable in the biological tissue such that the flexible probe and the rigid carrier are separable and the rigid carrier is removable from the biological tissue.

### BRIEF DESCRIPTION OF THE DRAWINGS

In the drawings, like reference characters generally refer to like parts throughout the different views. The drawings are not necessarily to scale, emphasis instead generally being placed upon illustrating the principles of the invention. In the following description, various embodiments of the invention are described with reference to the following drawings, in which:

FIG. 1A shows a schematic block diagram of a probe device, according to various embodiments.

FIG. 1B shows a flow chart illustrating a method of fabricating a probe device, according to various embodiments.

FIG. 1C shows a schematic block diagram of a probe device, according to various embodiments.

FIG. 2A shows a schematic perspective view of a probe device, according to various embodiments.

FIG. 2B shows a schematic perspective view of a probe device, according to various embodiments.

FIG. 2C shows a schematic of the probe device of the embodiment of FIG. 2B when in use at an insertion site, according to various embodiments.

FIGS. 3A to 3N show cross-sectional views of a process to fabricate a probe device, according to various embodiments.

FIGS. 4A and 4B show microscopy images of a silicon-PEG-silicon probe device before and after cortical implantation respectively.

FIG. 4C shows a microscopy image of a silicon-PEG-silicon probe device after cortical implantation.

FIG. 4D shows a microscopy image of a silicon-PEG-silicon probe device after submerging in artificial cerebrospinal fluid (a-CSF).

FIGS. 4E and 4F show microscopy images of a silicon-PEG-silicon probe device after cortical implantation on an unwetted cortical surface.

### DETAILED DESCRIPTION

The following detailed description refers to the accompanying drawings that show, by way of illustration, specific details and embodiments in which the invention may be practiced. These embodiments are described in sufficient detail to enable those skilled in the art to practice the invention. Other embodiments may be utilized and structural, logical, and electrical changes may be made without departing from the scope of the invention. The various embodiments are not necessarily mutually exclusive, as some embodiments can be combined with one or more other embodiments to form new embodiments.

Embodiments described in the context of one of the methods or devices are analogously valid for the other method or device. Similarly, embodiments described in the context of a method are analogously valid for a device, and vice versa.

Features that are described in the context of an embodiment may correspondingly be applicable to the same or similar features in the other embodiments. Features that are described in the context of an embodiment may correspondingly be applicable to the other embodiments, even if not explicitly described in these other embodiments. Furthermore, additions and/or combinations and/or alternatives as described for a feature in the context of an embodiment may correspondingly be applicable to the same or similar feature in the other embodiments.

In the context of various embodiments, the articles “a”, “an” and “the” as used with regard to a feature or element includes a reference to one or more of the features or elements.

In the context of various embodiments, the phrase “at least substantially” may include “exactly” and a reasonable variance.

In the context of various embodiments, the term “about” or “approximately” as applied to a numeric value encompasses the exact value and a reasonable variance.

As used herein, the term “and/or” includes any and all combinations of one or more of the associated listed items.

Various embodiments may relate to the field of microfabricated neural implants. Various embodiments may be related to long term chronic recording neural probes, and may be employed in applications related to long term chronic neural implant.

Various embodiments may provide a neural probe that may uncouple the requirements of a long term implanted probe, which may be flexible, from the specifications needed for minimally damaging insertion, that may require stiff, sharp, straight probes that may be inserted relatively fast but under control. Various embodiments may also enable drug delivery in the neural probe.

Various embodiments may provide a flexible polyethylene glycol (PEG) Parylene neural probe with a retractable silicon (Si) backbone, with drug delivery capability. Various embodiments may provide a microfabricated flexible Parylene based neural probe that may be attached to a silicon (Si) backbone through a temporary glue, for example Polyethylene glycol (PEG). In various embodiments, retraction of the stiff silicon (Si) backbone may be carried out upon dissolution of the PEG in a biological tissue, e.g. cortex. The Si backbone (stiff component) may be retracted in a hydrophilic environment. The neural probe may also enable drug delivery during dissolution of the temporary glue (e.g. PEG) during implantation.

Various embodiments may provide a structure including a flexible neural probe component with an embedded microchannel or recess, attached to a stiff retractable silicon (Si) backbone through a temporary glue, for example Polyethylene Glycol or PEG, which may also be used as an agent for drug delivery. The approach of various embodiments may provide the possibility of drug delivery through the dissolution of the temporary glue (e.g. PEG), a structure with an embedded microchannel, and lithographic definition and relative positioning of the microfabricated stiff and flexible portions of the neural probe. The method of various embodiments provides for the incorporation of the biomaterial, e.g. PEG, in the microfabrication of this flexible probe with a retractable backbone.

The approach of various embodiments may provide one or more of the following: 1) reduction of mechanical mismatch between the brain tissue and the probe, which may eventually lead to reduced inflammatory reaction and hence reduced encapsulation of the probe. Therefore, the design of various embodiments may address the challenges for a long term recording probe by uncoupling the requirements of a long term implanted probe (flexibility) from the specifications needed for minimally damaging insertion (stiff sharp straight probes that may be inserted relatively fast but under control); 2) having a stiff but retractable backbone to enable flexible probe penetration with the same ease as conventional silicon (Si) probe penetration; 3) integrated drug delivery upon dissolution of the temporary glue (biodegradable material); and 4) a design wherein biosafe materials may be left in the brain without the long term effects of stiff degradable insertion shuttles (possible examples include but are not limited to a porous Si or a bioglass based penetrating shank).

Various embodiments may further provide a method to integrate a temporary glue (e.g. PEG) in the microfabrication of a flexible neural probe with a stiff backbone, such that the flexible and stiff portions of the probe may be lithographically defined and positioned, relative to each other, for assembly of the flexible and stiff portions of the probe.

Various embodiments may provide an approach in engineering the fabrication to incorporate a biodegradable temporary glue to sandwich the stiff and flexible components of the neural probe.

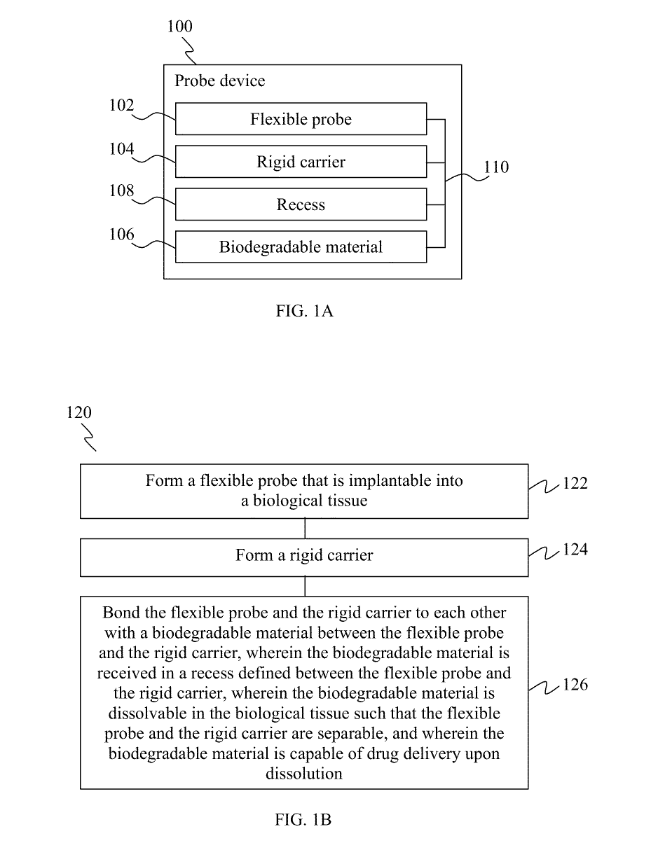

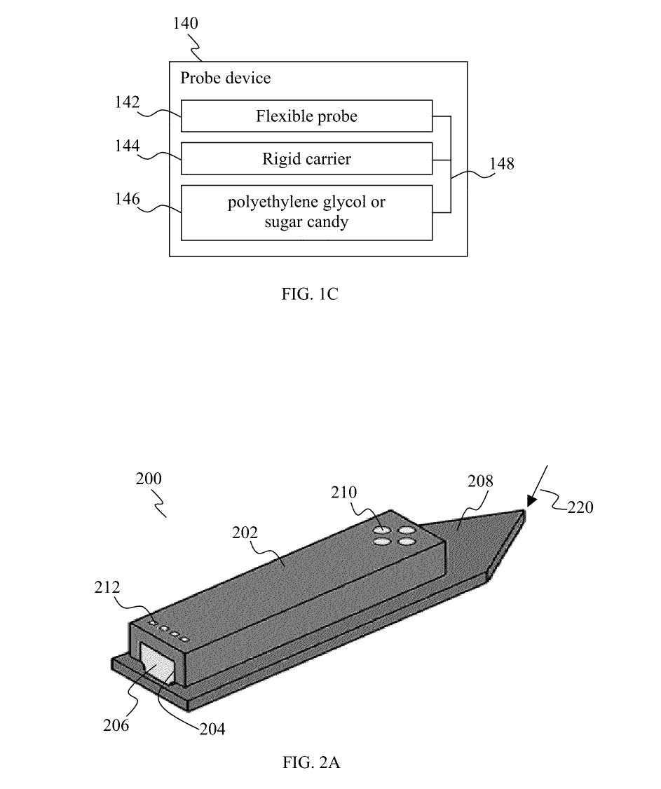

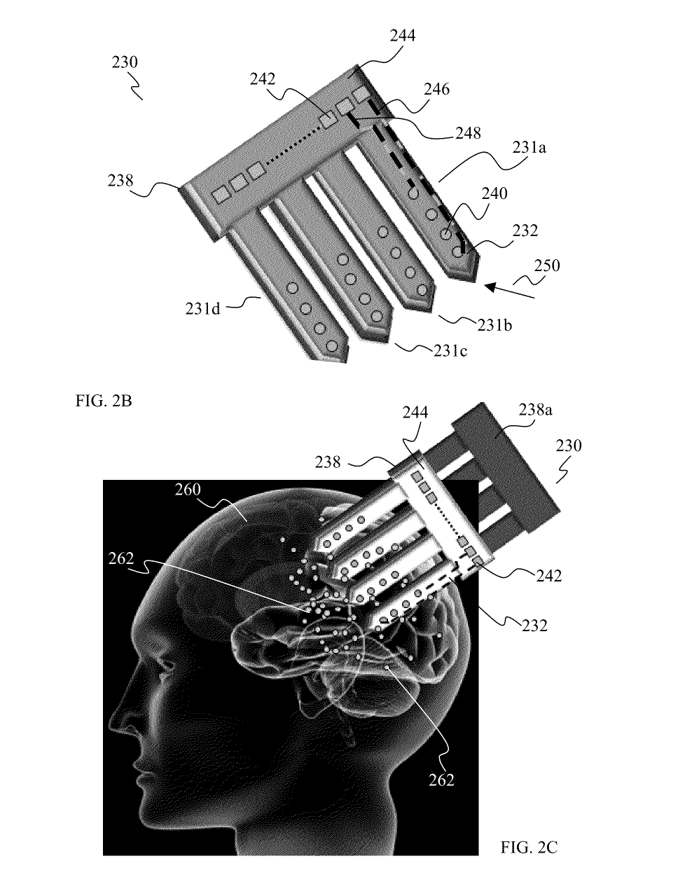

FIG. 1A shows a schematic block diagram of a probe device 100, according to various embodiments. The probe device 100 includes a flexible probe 102 that is implantable into a biological tissue, a rigid carrier 104, and a biodegradable material 106 received in a recess 108 defined between the flexible probe 102 and the rigid carrier 104, the biodegradable material 106 bonding the flexible probe 102 and the rigid carrier 104 to each other, wherein the biodegradable material 106 is dissolvable in the biological tissue such that the flexible probe 102 and the rigid carrier 104 are separable and the rigid carrier 104 is removable from the biological tissue, and wherein the biodegradable material 106 is capable of drug delivery upon dissolution. In FIG. 1A, the line represented as 110 is illustrated to show the relationship among the flexible probe 102, the rigid carrier 104, the biodegradable material 106 and the recess 108, which may include mechanical coupling.

In other words, a recess 108 is defined between the flexible probe 102 and the rigid carrier 104, in which the recess 108 contains a biodegradable material 106. The biodegradable material 106 acts as an adhesive or a bonding agent for bonding or attaching the flexible probe 102 and the rigid carrier 104 to each other. When the probe device 100 is implanted into a biological tissue (e.g. brain, cortex), the biodegradable material 106 may be dissolved upon contact with the biological tissue. As a result of the dissolution of the biodegradable material 106, the flexible probe 102 and the rigid carrier 104 may be detached from each other and the rigid carrier 104 may then be removed from the biological tissue, while the flexible probe 102 may remain in the biological tissue, for example for taking measurements relating to activities of the biological tissue.

In various embodiments, the recess 108 may be formed in a material of the flexible probe 102. However, it should be appreciated that the recess 108 may, additionally or alternatively, be formed in a material of the rigid carrier 104. In other words, the recess 108 may be formed, at least partially, through the respective materials of the flexible probe 102 and/or the rigid carrier 104.

In various embodiments, the biodegradable material 106 may further include drugs and/or steroids that may be releasable on the dissolution of the biodegradable material 106. As the biodegradable material 106 dissolves over time, the drugs and/or steroids may be released over a period of time.

In various embodiments, the flexible probe 102 may include at least one electrode arranged at an insertion end of the flexible probe 102, and at least one contact pad electrically coupled to the electrode, the contact pad being configured for electrical coupling to an external circuit. The insertion end of the flexible probe 102 means the end of the flexible probe 102 implantable into the biological tissue at an insertion or implantation site. The flexible probe 102 may further include at least one interconnection buried or embedded within the flexible probe 102, the interconnection electrically coupling the electrode and the contact pad.

In various embodiments, the flexible probe 102 may include a plurality of electrodes arranged at an insertion end of the flexible probe 102, and a plurality of contact pads, wherein a respective contact pad is electrically coupled to a respective electrode, and wherein the plurality of contact pads are configured for electrical coupling to an external circuit. The plurality of electrodes may be arranged spaced apart from each other. The plurality of electrodes may be arranged in a line. The plurality of electrodes may be aligned along a longitudinal axis of the flexible probe 102.

In the context of various embodiments, the probe device 100 may include a plurality of flexible probes 102, wherein the biodegradable material 106 is received in a respective recess 108 defined between a respective flexible probe 102 and the rigid carrier 104, the biodegradable material 106 bonding the respective flexible probe 102 and the rigid carrier 104 to each other.

In the context of various embodiments, a width of the flexible probe 102 may be between about 150 μm and about 200 μm, for example between about 150 μm and about 180 μm, between about 150 μm and about 160 μm or between about 175 μm and about 200 μm. In various embodiments, a width of the rigid carrier 104 may be at least substantially similar to the width of the flexible probe 102. This may enable a smaller penetration area at an insertion site.

In the context of various embodiments, the flexible probe 102 may have a Young's modulus of between about 360 kPa and about 4 GPa, for example between about 360 kPa and about 1 GPa, between about 360 kPa and about 500 MPa, between about 360 kPa and about 100 MPa, between about 360 kPa and about 1 MPa, between about 360 kPa and about 500 kPa, between about 1 MPa and about 4 GPa, between about 500 MPa and about 4 GPa, or between about 500 MPa and about 2 GPa.

In the context of various embodiments, the rigid carrier 104 may have a Young's modulus of between about 100 GPa and about 170 GPa, for example between about 100 GPa and about 150 GPa, between about 100 GPa and about 120 GPa, between about 120 GPa and about 170 GPa, or between about 120 GPa and about 150 GPa.

In the context of various embodiments, the biodegradable material 106 may be selected from the group consisting of polyethylene glycol (PEG), polysaccharide, sugar candy and any combination thereof.

In the context of various embodiments, the flexible probe 102 may include or may be made of a polymeric material. The polymeric material may be selected from the group consisting of Parylene, polyimide, polydimethylsiloxane, poly(methyl methacrylate) (PMMA), silicone, and SU-8.

In the context of various embodiments, the rigid carrier 104 may include or may be made of a material selected from the group consisting of silicon (Si), titanium (Ti), and glass.

FIG. 1B shows a flow chart 120 illustrating a method of fabricating a probe device, according to various embodiments.

At 122, a flexible probe that is implantable into a biological tissue is formed.

At 124, a rigid carrier is formed.

At 126, the flexible probe and the rigid carrier are bonded to each other with a biodegradable material between the flexible probe and the rigid carrier, wherein the biodegradable material is received in a recess defined between the flexible probe and the rigid carrier, wherein the biodegradable material is dissolvable in the biological tissue such that the flexible probe and the rigid carrier are separable, and wherein the biodegradable material is capable of drug delivery upon dissolution.

In various embodiments of the method, the recess may be formed in a material of the flexible probe.

In various embodiments, prior to bonding the flexible probe and the rigid carrier, a pair of sacrificial tracks may be formed between the flexible probe and the rigid carrier. The pair of sacrificial tracks may be removed after bonding the flexible probe and the rigid carrier.

The method may further include thermally bonding or microwave assisted bonding the flexible probe and the rigid carrier.

The method may further include adding drugs and/or steroids within the biodegradable material, the drugs and/or steroids being releasable on the dissolution of the biodegradable material.

In various embodiments of the method, the biodegradable material may be selected from the group consisting of polyethylene glycol (PEG), polysaccharide, sugar candy and any combination thereof.

FIG. 1C shows a schematic block diagram of a probe device 140, according to various embodiments. The probe device 140 includes a flexible probe 142 that is implantable into a biological tissue, a rigid carrier 144, and polyethylene glycol 146 or sugar candy 146 between the flexible probe 142 and the rigid carrier 144 bonding the flexible probe 142 and the rigid carrier 144 to each other, in which the polyethylene glycol 146 or the sugar candy 146 is dissolvable in the biological tissue such that the flexible probe 142 and the rigid carrier 144 are separable and the rigid carrier 144 is removable from the biological tissue. This means that the flexible probe 142 and the rigid carrier 144 may be bonded to each other by polyethylene glycol 146 or sugar candy 146 that is provided in between the flexible probe 142 and the rigid carrier 144. The polyethylene glycol 146 or the sugar candy 146 may be dissolvable in the biological tissue, for example as a result of contact with the biological tissue.

In FIG. 1C, the line represented as 148 is illustrated to show the relationship among the flexible probe 142, the rigid carrier 144, and the polyethylene glycol 146 or the sugar candy 146, which may include mechanical coupling.

The polyethylene glycol 146 or the sugar candy 146 may be received in a recess defined between the flexible probe 142 and the rigid carrier 144. The recess may be formed in a material of the flexible probe 142. However, it should be appreciated that the recess may, additionally or alternatively, be formed in a material the rigid carrier 144. In other words, the recess may be formed, at least partially, through the respective materials of the flexible probe 142 and/or the rigid carrier 144.

In the context of various embodiments, the flexible probe 142 may include or may be made of Parylene and the rigid carrier may include or may be made of silicon (Si).

In the context of various embodiments, the flexible probe 102, 142 may be deformable. The rigid carrier 104, 144 may be stiff and/or may be less flexible or deformable as compared to the flexible probe 102, 142.

In the context of various embodiments, the term “rigid carrier” may mean a stiff backbone or an insertion shuttle.

In the context of various embodiments, the term “recess” may mean a channel, for example a microchannel, or a void.

In the context of various embodiments, the term “biological tissue” may include a living tissue, for example brain tissue, neural tissue, or cortex.

Various embodiments may provide an approach that combines the ease of penetration similar to the traditional stiff silicon (Si) probes, but leaves a flexible probe in a biological tissue, e.g. cortex, after insertion of the flexible probe. Various embodiments may provide a flexible Parylene neural probe attached to a stiff silicon (Si) backbone using a temporary biodegradable glue, for example Polyethylene Glycol (PEG). In addition, the biodegradable glue or biodegradable material may be sugar candy, for example sugar candy with maltose or maltose candy.

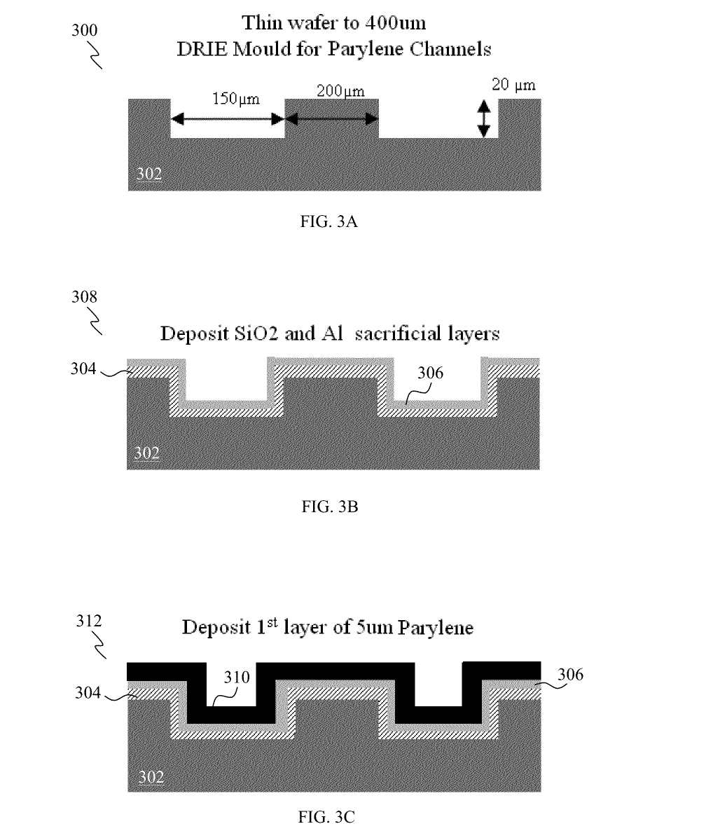

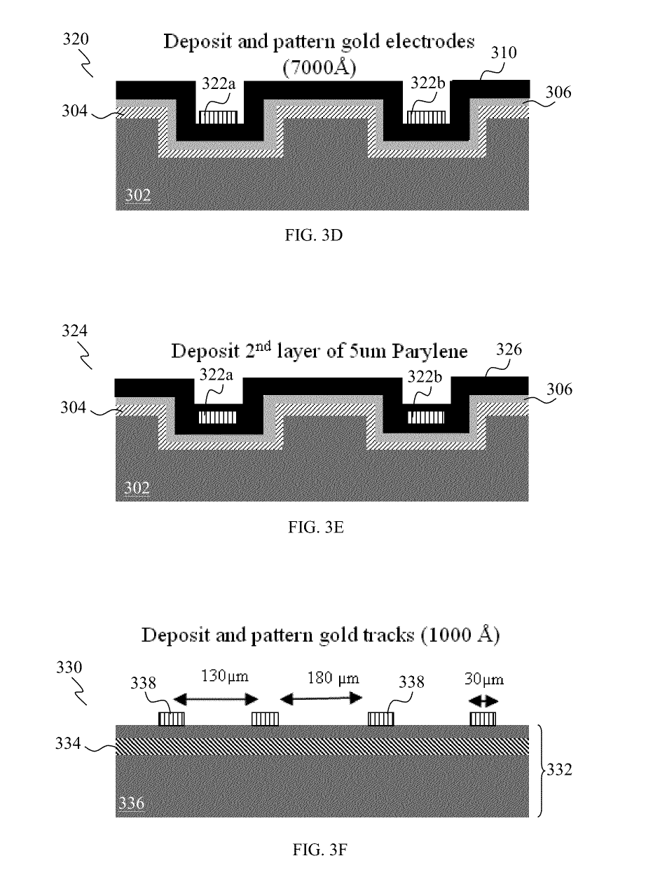

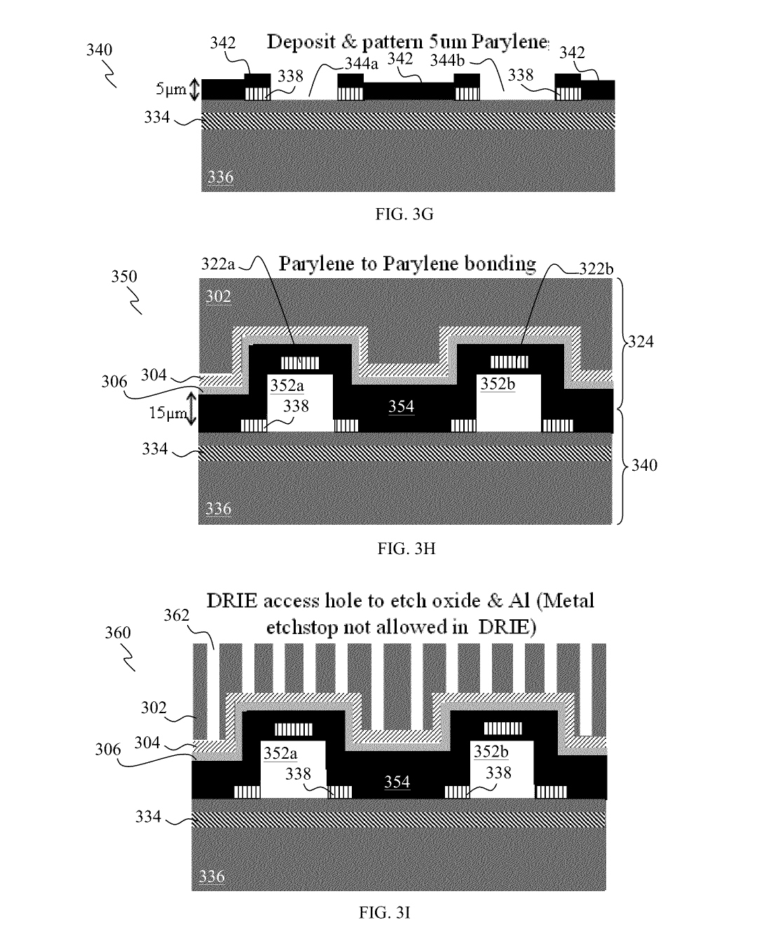

FIG. 2A shows a schematic perspective view of a probe device or neural probe 200, according to various embodiments. The probe device 200 has a single or individual probe shank. A non-limiting examples, the probe device 200 may be a flexible PEG-Parylene neural probe with a retractable silicon (Si) backbone, with drug delivery capability.

The probe device or neural probe 200 includes a structure having a flexible portion or probe 202, with a microfluidic channel or recess (e.g. an integrated microfluidic channel or recess) 204 that contains a biodegradable glue or biodegradable material 206, which may be PEG, and a stiff retractable backbone (e.g. Si backbone) or carrier 208. The probe device 200 may be implantable into a biological tissue, e.g. cortex.

The flexible probe 202 may have an at least substantially rectangular shape, with the recess 204 defined throughout the length of the flexible probe 202, or at least partially through the length of the flexible probe 202. The flexible probe 202 may be arranged on the backbone 208 such that the recess 204 is positioned between the flexible probe 202 and the backbone 208 such that the biodegradable material 206 contained, at least partially, within the recess 204 may contact the flexible probe 202 and the backbone 208 for bonding the flexible probe 202 and the backbone 208 to each other.

The flexible probe or component 202, for example made of Parylene, may include exposed electrodes (e.g. gold (Au) electrodes), for example as represented by 210 for one electrode, towards one end (e.g. distal end) of the flexible probe 202, and exposed bond pads or contact pads (e.g. gold (Au) contact pads), for example as represented by 212 for one contact pad, towards an opposite end (e.g. proximal end) of the flexible probe 202. The contact pads, e.g. 212 may be used for electrical coupling to an external circuit. The flexible probe 202 may further include one or more interconnects (not shown) covered by the material of the flexible probe 202, for example buried or embedded in the flexible probe 202, for electrical coupling between the electrodes (e.g. 210) and the contact pads (e.g. 212). Therefore, the flexible probe 202 may for example be a Parylene flex circuit.

The flexible probe 202 may be arranged or bonded to a portion of the backbone 208. The backbone 208 may have an at least substantially sharp end, as indicated by the arrow 220 in FIG. 2A, for facilitating the insertion or implantation of the probe device 200 and therefore also the flexible probe 202 into the biological tissue. Therefore, the sharp end of the backbone 208 may correspond to the insertion end of the probe device 200.

The biodegradable material 206, e.g. PEG, contained in the embedded microchannel or recess 204, may be a temporary glue, which may be dissolvable in the biological tissue which the probe device 200 is implanted into. Upon dissolution of the biodegradable material 206, the flexible portion 202 and the backbone 208 may become separable and the backbone 208 may be retracted from the biological tissue, leaving the flexible probe 202 implanted in the biological tissue. In addition, the biodegradable material 206 may carry drugs, in other words, drugs may be embedded or mixed within the biodegradable material 206, such that the biodegradable material 206 may be used to deliver drugs on dissolution of the biological material 206 in the biological tissue.

In various embodiments, the probe device 200 may be used for insertion into the cortex of a patient. The probe device 200 may include flexible Parylene insulation layers 202 with exposed Au electrodes (e.g. 210) connected to exposed contact pads (e.g. 212), which may remain in the cortex. The probe device 200 further includes a silicon (Si) backbone 208 for facilitating penetration of the probe device 200 into the cortex, thereby retaining the ease of penetration similar to the traditional stiff silicon (Si) probes. The probe device 200 further includes polyethylene glycol (PEG) 206 acting as a temporary adhesive holding the flexible Parylene-electrode-Parylene 202 to the stiff Si backbone 208. Upon dissolution of the PEG 206, the Si backbone 208 may be retracted, leaving only the Parylene flexible substrate 202 implanted in the cortex.

FIG. 2B shows a schematic perspective view of a probe device 230, according to various embodiments. As a non-limiting examples, the probe device 230 may be a flexible PEG-Parylene neural probe with a retractable silicon (Si) backbone, with drug delivery capability. The probe device 230 may be implantable into a biological tissue, e.g. cortex.

The probe device 230 may be an implantable unit, having a set of multiple shanks. The probe device 230 may include a first probe shank 231a, a second probe shank 231b, a third probe shank 231c and a fourth probe shank 231d, which may be connected to a common backend 244. However, it should be appreciated that any number of probe shanks may be provided, for example two, three, four, five or any higher number.

It should be appreciated that any one of or each of the first probe shank 231a, second probe shank 231b, third probe shank 231c and fourth probe shank 231d may include a flexible probe (for example made of Parylene), e.g. 232, as substantially described in the context of the flexible probe 202, and a biodegradable material or biodegradable glue as substantially described in the context of the biodegradable material 206. The probe device 230 may include a common backbone (for example silicon (Si) backbone) 238. The common backbone 238 may correspond to the shape and/or dimension of the first probe shank 231a, the second probe shank 231b, the third probe shank 231c, the fourth probe shank 231d, and the common backend 244. As illustrated in FIG. 2B, using the first probe shank 231a as a non-limiting example, the flexible probe 232 may have an at least substantially similar shape and dimension as the portion of the common backbone 238 corresponding to the first probe shank 231a.

Using the first probe shank 231a as a non-limiting example, the flexible probe 232 includes exposed electrodes (e.g. gold (Au) electrodes), for example as represented by 240 for one electrode. The electrodes, e.g. 240, may be arranged in a line, for example along a length of the flexible probe 232. The common backend 244 may include exposed bond pads or contact pads (e.g. gold (Au) contact pads), for example as represented by 242 for one contact pad. The contact pads 242 may be arranged in a line, for example along a length of the common backend 244. The contact pads, e.g. 242 may be used for electrical coupling to an external circuit.

The probe device 230 may further include one or more interconnects, for example as represented by dashed lines 246, 248 for two interconnects, which for example may be buried or embedded in the flexible probe 232, for electrical coupling between the electrodes (e.g. 240) and the contact pads (e.g. 242).

The first probe shank 231a, the second probe shank 231b, the third probe shank 231c, the fourth probe shank 231d, and the portions of the common backbone 238 corresponding to the first probe shank 231a, the second probe shank 231b, the third probe shank 231c, and the fourth probe shank 231d, may respectively have an at least substantially sharp end, for example as indicated by the arrow 250 relating to the first probe shank 231a, for facilitating the insertion or implantation of the probe device 230 into the biological tissue.

FIG. 2C shows a schematic of the probe device 230 of the embodiment of FIG. 2B when in use at an insertion site, according to various embodiments. The probe device 230 may be employed for implantation into a cortex 260. It should be appreciated that the dimensions of the probe device 230 are exaggerated relative to the cortex 260 for clarity purposes. FIG. 2C illustrates the probe device 230 in an initial state, where the common backbone 238 is bonded to the flexible probes, e.g. flexible probe 232 of the first probe shank 231a, via a biodegradable glue, and a subsequent state where, upon dissolution of the biodegradable glue, the common backbone, as represented by 238a, may be separated from the flexible probes and removed from the cortex 260. Also illustrated in FIG. 2C are drugs, as represented by the white circles and denoted as 262 for two such circles, which may be contained in the biodegradable glue and which may be released into the cortex 260 on dissolution of the biodegradable glue.

It should be appreciated that features as described in the context of the probe device 200 may be applicable to like features of the probe device 230, and vice versa. In addition, the use and process of implantation into a biological tissue as described in the context of the probe device 200 may be applicable to the probe device 230, and vice versa.

For various embodiments of the probe devices 200, 230, preliminary experiments of in vivo dissolution of PEG in the cortex showed a minimum dissolution time of about 20 minutes, which may be acceptable to neurosurgeons. In some embodiments, a dissolution time of about 40 minutes may also be achieved in vivo.

PEG may be used as a biodegradable material in the cortex. In various embodiments of the method of fabricating a probe device, the PEG may be included or incorporated in the final stages of the probe fabrication, which makes it possible to mix the PEG with anti-inflammatory drugs or anti-inflammatory steroids and use the dissolution of the PEG as a means of drug delivery after probe insertion, for example at the cortex, thereby providing an additional functionality to the probe devices of various embodiments, as compared to conventional probes.

The fabrication method of various embodiments may integrate the inclusion of biodegradable PEG with the microfabrication such that the stiff and flexible portions of the neural probe may be lithographically defined and positioned, thereby providing a functionality compared to the manual alignment for different portions of conventional neural probes. Furthermore, in various embodiments, in terms of footprint, the stiff and flexible components may have similar dimensions or widths, for example 200 μm wide. In addition, the fabrication approach of various embodiments may not rely on diffusion limited removal of sacrificial material to fill the PEG, thus avoiding the possibility of spurious contamination in the biological tissue such as the brain.

One of the conventional approaches may include a highly hydrophilic, electronegative, self-assembled monolayer (SAM) as the temporary glue. An insertion shuttle is dipped in this SAM before placing an additional component and this is done immediately prior to cortical insertion. The shuttle component is 2× wide compared to the additional component, resulting in a larger insertion foot print and consequently larger kill zone, and possibly increased tissue response. In another conventional approach, the biodegradable glue is specifically restricted to polysaccharide and different components are manually placed.

One conventional approach to the insertion of probes in the cortex includes using a bioinspired material with switchable stiffness from sea cucumber dermis. To keep the stiffness of the probe intact during implantation, the brain cannot be supplemented by moisture. This leads to surface of the brain drying slowly, accumulated dried blood on the brain surface, increase in the elastic modulus of the pia making probe insertion increasingly difficult with time and preventing multisite insertion. In contrast, the approach of various embodiments features wetting or supplementing the brain surface with moisture, for example saline or artificial cerebrospinal fluid (a-CSF), during and after penetration implantation as validated by neurosurgeons, and therefore does not suffer from the above limitations. No dried blood clotting may be observable on the brain surface when supplemented by moisture in preliminary in-vivo studies.

Another conventional approach features a magnetic insertion system for flexible electrode implantation, where a pulsed magnetic field was generated in a coil surrounding a glass pipette containing the electrode and successful implantation into the rat cortex was achieved using 480 and 560V charges. The ferromagnetic tip used was not biocompatible and the system was not adapted for MEMS fabrication. In contrast, the microfabricated approach of various embodiments for the insertion of a flexible neural probe with a retractable backbone features biocompatible materials in the brain.

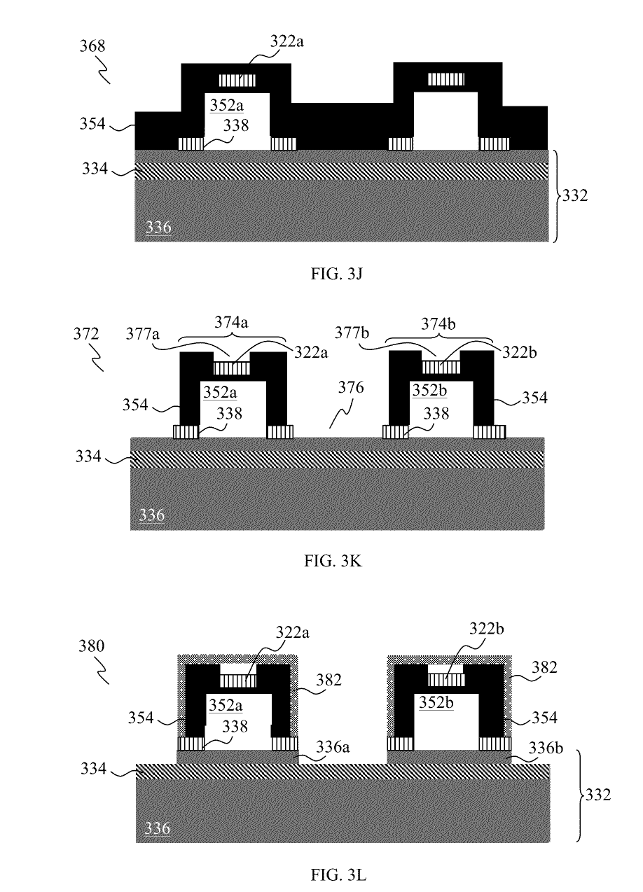

Various embodiments provide a method of integration of a temporary glue (e.g. PEG) in the microfabrication of a flexible neural probe with a retractable backbone. A non-limiting example of a fabrication process will now be described with reference to FIGS. 3A to 3N, illustrating one possible approach of fabricating a PEG Parylene probe with a stiff retractable Si backbone. It should be appreciated that while FIGS. 3A to 3N show a method for fabricating two probe shanks, the method may be employed to fabricate a probe device having any number of probe shank(s), for example four probes similar to the embodiment of FIG. 2B.

A silicon (Si) wafer is first provided and patterned in deep reactive-ion etching (DRIE) to create a mold for the flexible portion of the neural probe that would be formed later. FIG. 3A shows a structure 300 that may be obtained after the Si wafer or substrate 302 has been patterned and etched to create a mold for forming Parylene channels in a later process.

Next, as shown in FIG. 3B, sacrificial layers of silicon oxide (SiO₂) 304 and aluminum (Al) 306 are deposited in a plasma enhanced chemical vapor deposition (PECVD) tool and an evaporation tool respectively, thereby forming the structure 308.

A first layer 310 of Parylene may then be deposited or coated to a thickness of approximately 5 μm on the structure 308, resulting in the structure 312 ((FIG. 3C) being formed. Evaporation and patterning of gold (Au) electrodes, bond pads and interconnects may then be carried out via evaporation and etching processes. The Au electrodes, bond pads and interconnects may respectively have a thickness of about 7000 Å (700 nm). As a non-limiting example, FIG. 3D shows a structure 320 with two electrodes 322a, 322b.

Subsequently, a second layer of Parylene may be coated on the structure 320 to a thickness of approximately 5 μm. A structure 324 may be obtained, as shown in FIG. 3E. For ease of understanding and clarity, the first layer 310 of Parylene and the second layer of Parylene are collectively denoted as 326 in the structure 324. FIG. 3E also shows that the electrodes 322a, 322b are buried or embedded in the Parylene layer 326. Subsequently, the Parylene coated mold wafer, i.e. structure 324, may be set aside. The structure 324 may be used to form the flexible portion or component of the probe device of various embodiments.

Next, a silicon-on-insulator (SOI) wafer may be patterned to define sacrificial gold (Au) tracks of about 1000 Å thickness. FIG. 3F shows a structure 330 that may be obtained. The structure 330 includes a SOI wafer 332 with a silicon oxide (SiO₂) layer 334 buried in a silicon (Si) substrate 336, and four Au tracks, as represented by 338 for two such Au tracks. The Au tracks 338 may be used to hold the flexible and stiff components of the probe together until the PEG is inserted, as will be described later.

A Parylene layer of approximately 5 μm thick may then de deposited on the structure 330, including on the Au tracks 338, and anisotropically patterned using oxygen (O₂) plasma in reactive-ion etching (RIE), such that the Parylene is removed in the areas where the corresponding microchannels or recesses that contain the PEG is later formed. FIG. 3G shows a structure 340 that may be formed, having a patterned Parylene layer 342 and windows 344a, 344b, where Parylene has been anisotropically removed, which correspond to the areas where embedded microchannels may be later formed. The structure 340 may be used to form the stiff portion or component of the probe device of various embodiments.

Subsequently, the flexible Parylene component attached to the mold wafer may be aligned to the SOI wafer and thermally bonded at the Parylene-Parylene interface so as to form embedded microchannels or recesses. In other words, the structure 324 (FIG. 3E) and the structure 340 (FIG. 3G) may be aligned relative to each other and thermally bonded at the interface of the Parylene layer 326 of the structure 324 and the Parylene layer 342 of the structure 340, so as to attach or bond the fixed (stiff) and flexible portions of the probe device. As a result, as shown in FIG. 3H, a structure 350 may be obtained, having a first recess 352a and a second recess 352b. For ease of understanding and clarity, the Parylene layer 326 and the Parylene layer 342 are collectively denoted as 354 in the structure 350.

The bonded wafer or structure 350 may then be subjected to deep reactive ion etching (DRIE) on the mold wafer or structure 324 to open access to the sacrificial SiO₂ layer 304 and the sacrificial Al layer 306. As shown in FIG. 3I, a structure 360 may be obtained having access holes, as represented by 362 for one such access hole, formed through the thickness of the Si wafer or substrate 302.

Subsequently, the sacrificial SiO₂ layer 304 may be removed by vapor phase etching in a reactive ion etcher (RIE) (e.g. vapor phase oxide etch in Advance Vacuum (RIE)), and the sacrificial Al layer 306 may be removed by wet etching. This process releases and removes the mold wafer and exposes the Parylene-Au-Parylene sandwich bonded to the Parylene layer on the SOI wafer 332. As Parylene may be patterned through an aluminum (Al) mask, wet etching of the sacrificial Al layer 306 should not affect the Parylene layer 354. A structure 368 as shown in FIG. 3J may be obtained.

Next, the Parylene layer 354 may be anisotropically patterned and etched (e.g. via RIE) to define one or more flexible probe shanks or probe fingers and the backend space, and to open access to, and thereby exposing the Au electrodes 322a, 322b, and the bond pads, as well as to the Au tracks 338. The gold, for example the Au electrodes 322a, 322b, acts as an etch stop for the Parylene RIE process. As shown in FIG. 3K, a structure 372 may be obtained, where a first probe shank 374a and a second probe shank 374b may be defined, with the Parylene layer between the first probe shank 374a and the second probe shank 374b removed in the anisotropic patterning process, leaving an opening 376. Furthermore, the Parylene layer 354 corresponding to the first probe shank 374a and the second probe shank 374b has been etched to define respective openings 377a, 377b, to expose the respective electrodes 322a, 322b. Each of the first probe shank 374a and the second probe shank 374b may, individually or collectively, act as a probe device. For clarity purposes, the common backend that may be formed is not shown in FIG. 3K and the subsequent FIGS. 3L to 3N.

A photoresist may then be coated on the structure 372 and patterned so as to protect or cover the Parylene layer 354. Subsequently, DRIE may be carried out using the resist as a mask so as to pattern and define the retractable Si backbone or portion of the probe device of various embodiments. FIG. 3L shows a structure 380 that may be obtained, with a photoresist layer 382 covering the Parylene layer 354, and portions of the SOI wafer 332 not covered by the photoresist etched down to the buried SiO₂ layer 334, thereby leaving Si portions 336a, 336b that may function as the stiff retractable backbone or carrier.

Subsequently, buffered hydrofluoric acid (BHF) etching may be performed to etch the SiO₂ layer 334, where Parylene is at least substantially resistant to BHF. The BHF etching process facilitates individual probe release, and consequently an implantable unit or probe device may be released at a time, the probe device having a set of multiple probe shanks connected to a common backend. The photoresist layer 382 also protects the Parylene layer 354 during release of the individual neural probe structures during the buffered hydrofluoric acid (BHF) dip. A Piranha clean process may be integrated with the BHF etching. The photoresist 382 may then be removed.

Polyethylene glycol (PEG) may then be filled or injected into the embedded microchannels 352a, 352b for each of the first probe shank 374a and the second probe shank 374b, by capillary action. PEG is a liquid above 50° C. and is a solid at room temperature. FIG. 3M shows two individual probe shanks; the first probe shank 374a and the second probe shank 374b, each having PEG 386 filled in the first recess 352a and the second recess 352b respectively.

The last step of fabrication may include etching the temporary or sacrificial Au anchors or tracks 338 so that the stiff (Si portions 336a, 336b) and flexible (Parylene 354) components of the neural probe device 390 (FIG. 3N) may be attached through the biodegradable glue PEG only. PEG acts as a mask for Au etching with an etchant mixture of nitric acid (HNO₃):hydrochloric acid (HCl):DI water in the proportion 1:3:4 by volume. When in use, the PEG 386 dissolve in the cortex, thereby separating the stiff portion (Si portions 336a, 336b) and the flexible portion (Parylene 354) of the neural probe 390. Accordingly, the dissolution of the PEG 386 in the cortex may enable the retraction of the stiff component (Si portions 336a, 336b) of the neural probe 390 from the cortex.

Proof of concept experimental validation will now be described by way of the following examples, based on a sandwiched structure of two silicon (Si) probes bonded together by PEG (e.g. a Si-PEG-Si probe device). It should be appreciated that at least substantially similar results as those described below based on the Si-PEG-Si probe device may also be applicable to the silicon-PEG-Parylene probe device of various embodiments.

In order to test the separation of structures through dissolution of PEG in the cortex, two microfabricated Si neural probe structures or devices (multiple shanks connected to a common backend) may be glued together using PEG. After the PEG has solidified holding the Si-PEG-Si sandwich, the structure may be inserted into a rat cortex.

Male Sprague-Dawley rats (approximately 270-350 g by weight) were procured from the Laboratory Animal Care, Singapore and maintained in the Centre for Life Sciences Vivarium under standard housing conditions (12 hour light and dark cycle, Temperature approximately 23-25° C.). All experiments were carried out with Institutional Animal Care and Use Committee (IACUC) approval.

Rats were anesthetized with a freshly prepared cocktail of Ketamine and Xylazine. Their heads were shaved and the rats were mounted in a stereotaxic frame (Benchmark, USA). A midline sagittal incision was performed to expose the skull and bleeding was prevented using a hemostatic pencil.

Linear burr holes (Length: about 3-4 mm, Width: about 1 mm) were drilled over the right and left parietal regions. The dura was incised using a sterile needle to facilitate the smooth entry of the Si-PEG-Si probes. The exposed brain surface was periodically moistened with sterile isotonic saline or a-CSF. The probes were gradually lowered into the brain tissue using fine forceps and the probes were left in place for about 20 minutes or about 40 minutes, and then removed gradually. The probes were then examined under a light microscope. Following the procedure, the rat was sacrificed with an excess dose of pentobarbital administered intraperitoneally.

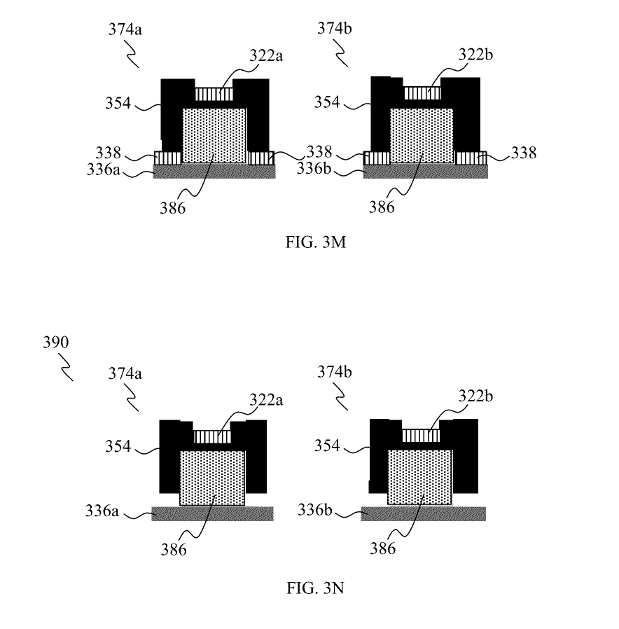

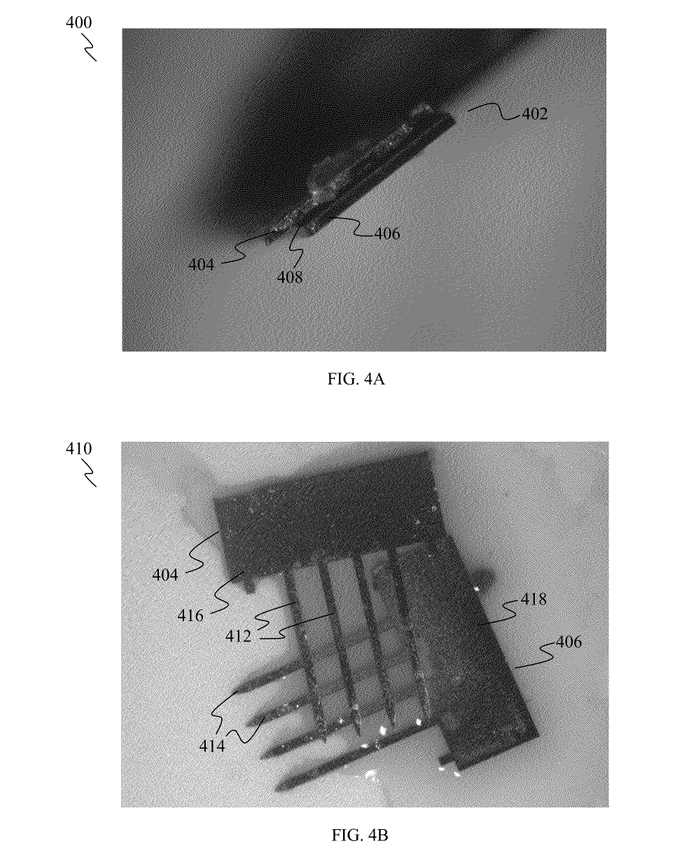

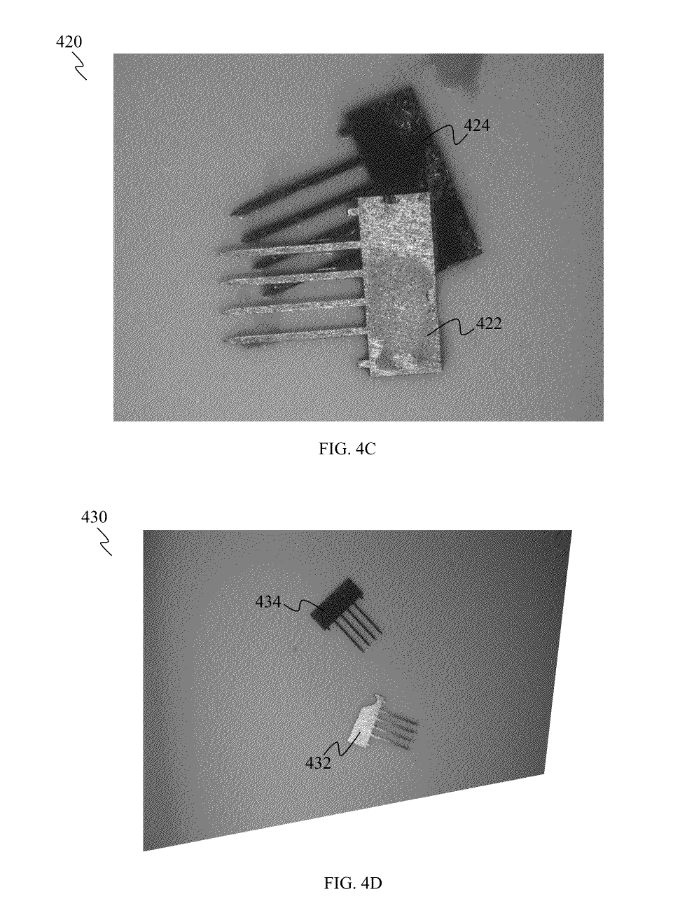

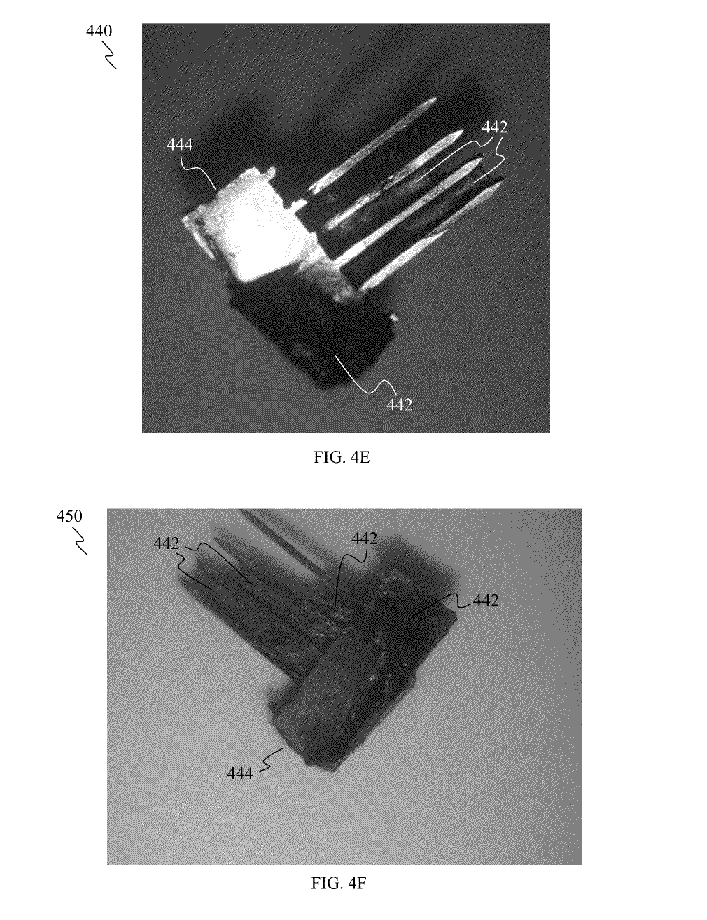

It was found that the explanted probes were separated upon removal from the cortex after 20 minutes of insertion (see for example FIG. 4B), indicating complete dissolution of the temporary glue PEG in the cortex. This procedure was verified by neurosurgeons and a dissolution time of 20 minutes was found to be acceptable. Further optimizations to reduce the PEG dissolution time, for example varying the amount and/or composition of PEG, and/or varying the surface of the PEG exposed as the dissolution time is proportional to the surface exposed, may be carried out.

FIGS. 4A and 4B show microscopy images of a silicon-PEG-silicon probe device before (image 400, FIG. 4A) and after (image 410, FIG. 4B) cortical implantation respectively.

FIGS. 4A and 4B illustrate the separation of silicon-PEG-silicon sandwiched probes after cortical implantation, where the image 400 illustrates a sandwiched Si-PEG-Si probe 402 (from the backview looking at the respective common backends) having a first Si probe 404 and a second Si probe 406 with PEG 408 in between, attaching the first Si probe 404 and the second Si probe 406, before implantation in a rat cortex (Male Sprague-Dawley rats, 270-350 g), while the image 410 illustrates the first Si probe 404 and the second Si probe 406, from the sandwiched probe 402, separated on explantation or extraction, after 20 minutes of implantation in the cortex. For the implantation, the cortical surface was kept moist by squirting a-CSF on the brain surface. It should be appreciated that the cortical surface may also be kept moist by a supply of saline.

The probe device 402 was implanted for about 20 minutes. The probe device 402, including the first Si probe 404 with shanks, e.g. 412, and the second Si probe 406 with shanks, e.g. 414, were inserted into the cortex, such that the shanks 412, 414 were completely immersed, with the common backend 416 of the first Si probe 404 and the common backend 418 of the second Si probe 406 above the cortical surface, sitting on the cortical surface. The common backends 416, 418, and the brain were flooded with a-CSF. Saline or a-CSF is used routinely in rat brain surgery. The implantation procedure was approved by neuroscientists.

FIG. 4C shows a microscopy image 420 of a silicon-PEG-silicon probe device after cortical implantation, where the probe device includes a first Si probe 422 and a second Si probe 424 bonded to each other by PEG. The image 420 illustrates the first Si probe 422 and the second Si probe 424, separated upon explantation or extraction, after 40 minutes of implantation in the cortex (Male Sprague-Dawley rats, 270-350 g).

For the implantation, the silicon-PEG-silicon probe, including the first Si probe 422 and the second Si probe 424, was inserted fully into the cortical surface such that the probe device was not visible when implanted. The cortical surface was kept moist by providing a-CSF on the brain surface, although saline may alternatively be used.

A silicon-PEG-silicon probe device was also submerged in artificial cerebrospinal fluid (a-CSF). FIG. 4D shows a microscopy image of a silicon-PEG-silicon probe device, including a first silicon (Si) probe 432 and a second silicon (Si) probe 434, after submerging in artificial cerebrospinal fluid (a-CSF) for a time period of less than 15 minutes (<15 mins). As shown in FIG. 4D, the first Si probe 432 and the second Si probe 434 of the silicon-PEG-silicon probe device were floated apart after submersion in the a-CSF solution.

In another experiment, the probes were implanted in the brain without moisturizing the cortex with saline. FIGS. 4E and 4F show microscopy images of a silicon-PEG-silicon probe device, as viewed from opposite sides of the probe device, after cortical implantation on an unwetted cortical surface, illustrating the effects of probe implantation without moistening the brain surface structure.

During surgery and implantation, the brain was not supplemented by moisture. As shown in the image 440 (FIG. 4E) and the image 450 (FIG. 4F), signs of blood clotting 442, after implantation, may be observed. The formation of blood clots on the brain and the blood clots 442 prevented the Si-PEG-Si sandwich probe device 444 from coming apart or separating, after implantation, as the blood clots 442 hold the Si-PEG-Si sandwich of the probe device 444 together post extraction as a result of unwetted brain surface.

The procedure of various embodiments, including supplementing the brain with moisture, follows normal protocol for rat brain surgery and overcomes or addresses the problems associated with the conventional dry cortical insertion method.

In various embodiments, the dissolving component (biodegradable material) may be the biodissolvable glue PEG, and therefore longer term effects of the remnants or effects of a larger volume shank dissolution may not be a concern.

The approach of various embodiments may provide one or more of the following: (1) a simple manual insertion similar to conventional silicon neural probes; (2) a reduction of mechanical mismatch between the brain tissue and the probe device that may lead to reduced inflammatory reaction and hence reduced encapsulation of the probe; (3) a stiff retractable backbone that may enable easy penetration similar to conventional silicon (Si) neural probes while reducing the probe-tissue mismatch post implantation; (4) long term effects of degradable insertion shuttles may not be a significant issue as only the temporary glue (e.g. PEG) is bio-dissolved in the brain, as compared to, for example, conventional biodegradable backbone/dissolving core based neural probes; (5) integrated drug delivery mechanism upon dissolution of temporary glue (e.g. PEG); (6) multisite insertion while avoiding or minimising blood clots using the method of various embodiments; (7) a method of integration of temporary glue (e.g. PEG) in the microfabrication of a flexible neural probe with a retractable stiff backbone; (8) lithographic definition and relative positioning of the microfabricated flexible and stiff portions of the probe through PEG; (9) there is no dry environment requirement during fabrication, and therefore the probe may be fabricated using normal semiconductor clean room fabrication protocol; (10) batch fabrication of the probe devices; (11) possibility of drug delivery through PEG as PEG is incorporated in the last steps of the batch fabrication process; (12) all materials left in the brain after implantation and retraction of the stiff component are biocompatible and may be used in neural tissue; (13) MEMS fabrication may be possible using biocompatible materials; and (14) no or minimal spurious contamination in the brain, for example due to remnant materials resulting from removal of sacrificial matter during fabrication.

Various embodiments may also incorporate transdural penetration, for example by attaching the retractable component to an ultrasonic penetration device, where tip velocities of about 50 m/s may be achieved with ultrasonic penetration. Transdural penetration may provide the ability to implant probes without removing the meningeal layers, thereby simplifying surgical procedures and lessening trauma to cortical neurons. Furthermore, transdural implantation may also lessen the amount of tissue micromotion experienced by the brain, where a reduced micromotion of neural tissue may translate to reduced interfacial stress and glial scar formation. Accordingly, various embodiments may provide an approach for a minimally invasive point and shoot device, and/or with transdural penetration capability and/or minimally damaging insertion leaving a flexible probe in the cortical tissue.

Various embodiments may provide a flexible neural probe with a retractable backbone that uncouples the requirements of long term recording with the requirements for minimally damaging insertion. In addition, the embedded microchannel (or recess) of the flexible neural probe, filled with a temporary biodegradable glue, for example polyethylene glycol (PEG), may serve as an agent for drug delivery at the insertion site. Therefore, various embodiments may incorporate the PEG in the microfabrication of the enhanced neural probe. As discussed above, preliminary experimental data on separation of silicon (Si) structures glued together with PEG in the rat cortex showed encouraging proof-of-concept results. The implantation procedure is similar to conventional neural probe insertion surgery in rats and has been approved by neurosurgeons. Saline or a-CSF, which is routinely used in rat brain surgery, may be used to wet the brain surface. PEG dissolvability in the rat cortex was verified for 20 minutes and 40 minutes. Preliminary in-vivo results with PEG based separation of the neural probes of various embodiments in the rat cortex are very encouraging. In vivo testing in the rat cortex may be performed to record extracellular action potentials.

In various embodiments, the implantation procedure for the Si-PEG-Parylene based design may follow the normal implantation procedure for rat brain surgery and may overcome or at least address problems associated with dry cortical insertion methods and blood clotting. The procedure allows for multisite probe implantation without toughening of the pia.

In various embodiments, a target penetration force for porous Si design based on simulation result may be approximately 30 mN. Implantation of the probe devices of various embodiments may be carried out by hand.

While the invention has been particularly shown and described with reference to specific embodiments, it should be understood by those skilled in the art that various changes in form and detail may be made therein without departing from the spirit and scope of the invention as defined by the appended claims. The scope of the invention is thus indicated by the appended claims and all changes which come within the meaning and range of equivalency of the claims are therefore intended to be embraced.

While the preferred embodiments of the devices and methods have been described in reference to the environment in which they were developed, they are merely illustrative of the principles of the inventions. The elements of the various embodiments may be incorporated into each of the other species to obtain the benefits of those elements in combination with such other species, and the various beneficial features may be employed in embodiments alone or in combination with each other. Other embodiments and configurations may be devised without departing from the spirit of the inventions and the scope of the appended claims.

1. A probe device comprising:
a flexible probe that is implantable into a biological tissue;
a rigid carrier; and
a biodegradable material received in a recess defined between the flexible probe and the rigid carrier, the biodegradable material bonding the flexible probe and the rigid carrier to each other, wherein the biodegradable material is dissolvable in the biological tissue such that the flexible probe and the rigid carrier are separable and the rigid carrier is removable from the biological tissue, and wherein the biodegradable material is capable of drug delivery upon dissolution.

2. The probe device of claim 1, wherein the recess is formed in a material of the flexible probe.

3. The probe device of claim 1, wherein the biodegradable material is selected from the group consisting of polyethylene glycol, polysaccharide, sugar candy and any combination thereof.

4. The probe device of claim 1, wherein the flexible probe comprises a polymeric material.

5. The probe device of claim 4, wherein the polymeric material is selected from the group consisting of Parylene, polyimide, polydimethylsiloxane, poly(methyl methacrylate), silicone, and SU-8.

6. The probe device of claim 1, wherein the rigid carrier comprises a material selected from the group consisting of silicon, titanium, and glass.

7. The probe device of claim 1, wherein the biodegradable material further comprises drugs and/or steroids that are releasable on the dissolution of the biodegradable material.

8. The probe device of claim 1, wherein the flexible probe comprises:
at least one electrode arranged at an insertion end of the flexible probe; and
at least one contact pad electrically coupled to the electrode, the contact pad being configured for electrical coupling to an external circuit.

9. The probe device of claim 8, wherein the flexible probe further comprises at least one interconnection buried within the flexible probe, the interconnection electrically coupling the electrode and the contact pad.

10. The probe device of claim 1, wherein the probe device comprises a plurality of flexible probes, wherein the biodegradable material is received in a respective recess defined between a respective flexible probe and the rigid carrier, the biodegradable material bonding the respective flexible probe and the rigid carrier to each other.

11. A method of fabricating a probe device, the method comprising:
forming a flexible probe that is implantable into a biological tissue;
forming a rigid carrier; and
bonding the flexible probe and the rigid carrier to each other with a biodegradable material between the flexible probe and the rigid carrier, wherein the biodegradable material is received in a recess defined between the flexible probe and the rigid carrier, wherein the biodegradable material is dissolvable in the biological tissue such that the flexible probe and the rigid carrier are separable, and wherein the biodegradable material is capable of drug delivery upon dissolution.

12. The method of claim 11, wherein the recess is formed in a material of the flexible probe.

13. The method of claim 11, further comprising:
forming a pair of sacrificial tracks between the flexible probe and the rigid carrier prior to bonding the flexible probe and the rigid carrier.

14. The method of claim 13, further comprising:
removing the pair of sacrificial tracks after bonding the flexible probe and the rigid carrier.

15. The method of claim 11, further comprising thermally bonding or microwave assisted bonding the flexible probe and the rigid carrier.

16. The method of claim 11, further comprising:
adding drugs and/or steroids within the biodegradable material, the drugs and/or steroids being releasable on the dissolution of the biodegradable material.

17. A probe device comprising:
a flexible probe that is implantable into a biological tissue;
a rigid carrier; and
polyethylene glycol or sugar candy between the flexible probe and the rigid carrier bonding the flexible probe and the rigid carrier to each other, in which the polyethylene glycol or the sugar candy is dissolvable in the biological tissue such that the flexible probe and the rigid carrier are separable and the rigid carrier is removable from the biological tissue.

18. The probe device of claim 17, wherein the polyethylene glycol or the sugar candy is received in a recess defined between the flexible probe and the rigid carrier.

19. The probe device of claim 18, wherein the recess is formed in a material of the flexible probe.

20. The probe device of claim 17, wherein the flexible probe comprises Parylene and the rigid carrier comprises silicon.
